(19) United States
(12) Patent Application Publication
(10) Pub. No.: US 2016/0067211 A1
(43) Pub. Date: Mar. 10, 2016

(54) PHARMACEUTICAL COMPOSITION

(71) Applicant: Eberhard Karls Universitaet Tuebingen Medizinische Fakultaet, Tuebingen (DE)

(72) Inventors: Christian Busch, Tuebingen (DE); Sascha Venturelli, Todtmoos (DE)

(21) Appl. No.: 14/925,859

(22) Filed: Oct. 28, 2015

Related U.S. Application Data
(63) Continuation of application No. PCT/EP2014/056464, filed on Mar. 31, 2014.

(30) Foreign Application Priority Data
Apr. 29, 2013 (DE) 10 2013 104 342.9

(51) Int. Cl. A61K 31/353; A61K 31/404; A61K 9/00; A61K 45/06

(52) U.S. Cl. CPC A61K 31/353; A61K 45/06; A61K 31/404; A61K 9/0014

(57) ABSTRACT

The present invention relates to a pharmaceutical composition for the prophylaxis and/or treatment of a melanoma and a precursor thereof as well as a skin and mucosa metastasis and uses and methods associated therewith.

### CROSS-REFERENCES TO RELATED APPLICATIONS

This application is a continuation of copending International Patent Application PCT/EP2014/056464 filed on Mar. 31, 2014 and designating the United States of America, which was not published under PCT Article 21(2) in English, and claims priority of Germany Patent Application DE 10 2013 104 342.9 filed on Apr. 29, 2013, which are both incorporated herein by reference.

### FIELD

The present invention relates to a pharmaceutical composition for the prophylaxis and treatment of a melanoma or a precursor thereof, as well as a skin or mucosa metastasis, and uses and methods associated therewith.

### BACKGROUND

The malignant melanoma is a highly malignant tumor of the pigment cells, the so-called melanocytes. It tends to spread metastases via the lymphatic and blood circulation at an early stage and is the most frequently lethal skin disease with a worldwide rapidly growing number of new cases.

Mostly the melanoma develops from a precursor. This group of cells already contains melanoma cells, which however are locally confined at the upper tissue layer. It is also referred to a melanoma in situ. One of the precursors of the malignant melanoma is defined as Lentigo maligna.

In the literature a distinction is drawn between at least five different subtypes of the malignant melanoma. The most frequent form is the superficial spreading melanoma (SSM). The most aggressive form of the malignant melanoma with the most unfavorable prognosis is the nodular malignant melanoma (NMM). Further subtypes are the lentigo maligna melanoma (LMM), the acral lentiginous melanoma (ALM) and the amenalotic melanoma (AMM).

Melanomas can metastasize into different organs, preferred target organs such as for other tumors do not exist. Very often metastases can be found in the liver, in the skin, in the lung, in the skeleton and in the brain.

Skin metastasis, also referred to as secondary skin cancer or metastatic skin cancer, is defined as a lymphogenic or hematogenic sealing of primary skin malignomas or tumors of other organs in the skin. As causal primary tumors in particular the following tumors come into questions in descending order: The malignant melanoma, mamma carcinoma, gestric carcinoma, uterus carcinoma, bronchial carcinoma, rectal carcinoma or renal carcinoma. They can appear anywhere on the body surface, preferably on the abdominal wall, the trunk and the capillitium.

### RELATED PRIOR ART

The most important form of therapy of the melanoma is still the surgical removal of the primary tumor and the excision of the metastases. Only the complete removal of the primary tumor at an early stage can result in healing. At later stages, where the tumor has already formed metastases in the skin, lymph nodes and internal organs, the chance for cure is low. This is where a good number of therapy alternatives are applied and tested which usually only provide a temporary recovery, however in most cases do not have a prospect of cure. These include chemotherapy with DTIC or fotemustin, a vaccination therapy with antigen presenting cells, surgical interventions to reduce the tumor mass, or a radiation therapy. More recent approaches of therapy are based on the blockade of molecular processes in the signal transduction of the cell. Here, for example, combinations of a traditional chemotherapeutic agent with b-RAF Kinase inhibitors such as sorafenib are used.

A very common form of the treatment of skin metastases is also the intralesional injection with Interleukin-2 (IL-2).

The surgical removal of the melanoma, its precursors or metastases is not always possible. In addition, the intervention is burdensome for the patient, painful after the operation, time-consuming and logistically complicated, in particular when there are long distances to the clinic or a critical general condition of the patient. Furthermore, the intervention is associated with scar formation and generally bears the risk of wound infections.

The intralesional injection of IL-2 also has significant disadvantages. It has to be carried out experimentally under study conditions over a longer period of time, three times a week in a skin center. Besides the high expenditure and the costs associated therewith this option of treatment is very often accompanied by severe and in parts systemic side effects. The application as such is painful and bears the risk of skin infections. Furthermore the IL-2 injection is only appropriate for skin metastases with a diameter of <5 mm and has only moderate response rates.

Because of the strongly increasing number of new cases and the therapeutic approaches which are so far not satisfying there is a strong need for new treatment options.

### SUMMARY OF THE INVENTION

Against this background it is an object of the present invention to provide a new pharmaceutical composition, by means of which a melanoma and a melanoma precursor as well as a skin and mucosa metastasis of any kind of primary tumor can be treated or prevented, respectively.

This object is met by the provision of a pharmaceutical composition which comprises as an active agent a prenylflavonoid, preferably prenylnaringenin (PN).

Prenylflavonoids belong to the group of flavonoids, a large group of lower molecular weight polyphenolic compounds. Flavonoids can be found in plants and consist of flavones, flavonoles, flavonones, flavan-3-oles and anthocyanines. The secondary plant metabolites play a role in the defense of the plant against micro-organisms or fungi and the protection against oxidative stress.

An important representative of the prenylflavonoids is the prenylnaringenin (PN).

It is understood that the pharmaceutical composition according to the invention can comprise a pharmaceutically acceptable formulation. Pharmaceutically acceptable formulations are well known in the state of the art. Exemplarily, it is referred to the essay of Kibbe A. (2000), Handbook of Pharmaceutical Excipients, 3rd edition, American Pharmaceutical Association and Pharmaceutical Press. The pharmaceutical composition according to the invention may also comprise additives. They include any compound or composition which is beneficial for the use according to the invention, which encompass salts, binding agents, solvents, dispersing agents and additional compounds which are usually applied in the formulation of medicinal products.

The object underlying the invention is herewith completely met.

According to an embodiment of the invention the pharmaceutical composition comprises 6-Prenylnaringenin (6-PN) and/or 8-Prenylnaringenin (8-PN).

6-PN and 8-PN are so-called prenylflavonoids. The can be found in low concentrations, e.g. in hops and in beer. The structure of this class of flavonoids is derived from 2-phenylchrom-4-on. 6-PN and 8-PN are isomers. The difference is in the position of the prenyl group consisting of five carbon atoms. 6-PN and 8-PN are products of a ring closure reaction of a common precursor molecule, desmethylxanthohumol, a polyphenol, which is based on the structure of 1,3-diphenyl-2-propene-1-one. Both molecules exist as enantiomeres (R, S).

8-PN has a strong binding affinity for estrogen receptors of the rat uterus and has been identified as a potent phytoestrogen due to a characteristic spacing of the hydroxyl groups, which imitate beta-estradiol; cf. Milligan et al. (1999), Identification of potent phytoestrogen in hops (Humulus lupulus L.) and beer, J. Clin. Endocrinol. Metab. 84, 2249-2252.

Apart from the antioxidative properties some flavonoids also have anticancer properties; cf. Hodek et al. (2002), Flavonoids-potent and versatile biologically active compounds interacting with cytochromes p. 450, Chem. Biol. Interact. 139, 1-21; Son et al. (2007), Pomiferin, histone deacetylase inhibitor isolated from fruits of Maclura pomifera, Bioorg. Med. Chem. Lett., Vol. 17(17), 4753-4755; Cidade et al. (2001), Artelastocarpin and carpelastofuran, two new flavones, and cytotoxicities of prenylflavonoids from Artocarpus elasticus against three cancer cell lines, Planta. Med. Vol. 67(9), 867-870; Kuete et al. (2011), Cytotoxicity and mode of action of four naturally occurring flavonoids from the genus Dorstenia: gancaonin Q, 4-hydroxylonchocarpin, 6-prenylapigenin, and 6,8-diprenyleriodictyol, Planta. Med. Vol. 77(18), 194-1989 (abstract).

8-PN inhibits the angiogenesis, induced by the basic fibroblast growth factor and the vascular endothelial growth factor in a three-dimensional collagen gel in vitro and in chorioallantois membrane assays in vivo; cf. Pepper et al. (2004), 8-Prenylnaringenin, A novel phytoestogen, inhibits angiogenesis in vitro and in vivo, J. Cell Physiol. 199, 98-107.

8-PN imitates the effects of 17β-Estradiol on the breast cancer cells MCF-7; cf. Rong et al. (2001), 8-Prenylnaringenin, the phytoestrogen in hops and beer, upregulates the function of the e-cadherin-/catenin complex in human mammary carcinoma cells, Eur. J. Cell Biol. 80, 5 580-585.

8-PN further induces the apoptosis in MCF7-cells and in a leukemia blast; cf. Brunelli et al. (2007), 8-Prenylnaringenin, inhibits estrogen receptor-α mediated cell growth and induces apoptosis in MCF-7 breast cancer cells, J. Steroid. Biochem. Mol. Biol. 107, 140-148, and Diller et al. (2007), Ability of prenylflavanones present in hops to induce apoptosis in a human Burkitt lymphoma cell line, Planta Med. 73, 755-761.

Recently also described was the inhibition of the P-glycoprotein, the transporter protein associated with multi resistency, and the inhibition of MRP1 by 8-PN; cf. Wesolowska et al. (2010), 8-Prenylnaringenin is an inhibitor of multidrug resistance-associated transporters, P-glycoprotein and MRP1, Eur. J. Pharmacol. 644, 32-40.

In addition, 8-PN directly inhibits the activation of the PI (3) K/Akt-signal path in MCF-7 cells in vitro; Brunelli et al. (2009), 8-Prenylnaringenin inhibits epidermal growth factor-induced MCF-7 breast cancer cell proliferation by targeting phosphatidylinositol-3-OH kinase activity, J. Steroid. Biochem. Mol. Biol. 113, 163-170.

6-PN and 8-PN show antiproliferative effects on the human prostate cancer cell lines PC-3 and DU145 in vitro; Delmulle et al. (2006), Anti-proliferative properties of prenylated flavonoids from hops (Humulus lupulus L.) in human prostate cancer cell lines, Phytomedicine 13, 732-734. This happens in the absence of a Caspase-3 activation and typical apoptotic morphological characteristics; Delmulle et al. (2008), Treatment of PC-3 and DU145 prostate cancer cells by prenylflavonoids from hop (Humulus lupulus L.) induces a caspase-independent form of cell death, Phytother. Res. 22, 197-203.

The suitability of 6-PN and 8-PN for the prophylaxis and treatment of a melanoma or a precursor thereof as well as a skin or mucosa metastasis of any primary tumor is so far not described in the state of the art or rendered obvious. This is also because the exact cellular mechanisms of 6-PN and 8-PN are not known so far.

The inventors were now able to demonstrate for the first time in silico and in vitro that 6-PN and 8-PN are distinct potent inhibitors of histone deacetylase (HDACs) of the classes I, II and IV and induce a hyperacetylation of the histone protein H3. This could be shown by the inventors using the example of different tumor cell lines, in particular by means of both of the melanoma cell lines SK-MEL-28 and BLM.

In addition, the inventors were able to demonstrate in a large number of tumor cell lines that 6-PN and 8-PN show strong apoptosis independent antiproliferative properties in vitro and low toxicity in vivo. In human epidermal skin reconstructions both PNs show a strong inhibited cell proliferation and invasion of the tumor cells.

Furthermore, the inventors were able to observe the induction of autophagy in tumor cells after the treatment with 6-PN and 8-PN.

Taken together, the data generated by the invention show that 6-PN and 8-PN are potent phytotherapeutic epigenetic active agents for the melanoma prevention, melanoma therapy, treatment and prophylaxis of skin and mucosa metastasis of any primary tumor.

According to an embodiment of the invention the pharmaceutical composition is configured for the prophylaxis and/or treatment of a melanoma or a melanoma precursor of the skin.

This measure has the advantage that the medicinal product is provided for the treatment of the most prevalent melanoma form, namely the cutaneous melanoma and its precursors. So far satisfying therapeutic approaches against such clinical pictures do not exist in the state of the art.

According to another embodiment of the invention the pharmaceutical composition is configured for the prophylaxis and/or treatment of melanoma skin and/or melanoma mucosa metastasis.

Also with this measure the treatment of such metastasis is addressed in a preferred manner for which so far no sufficient concepts of treatment do exist.

According to an embodiment of the invention the pharmaceutical composition is configured for a topical application, further preferably for a topical application onto the skin.

This measure has the advantage that for the first time an effective topical therapy of the melanoma, its precursors or of metastases of any primary tumor is possible, which can be carried out by the patient itself. For the patient it is far less burdensome, both physically as well as mentally, as current forms of therapy, for example the injection of the lesions with IL-2. Compared to surgical procedures and the IL-2 injection also the cost expenditure of such a topical therapy is significantly reduced. Also the risk of infections is strongly diminished. The topical configuration of the composition according to the invention furthermore allows the treatment of a larger skin area. According to findings of the inventors a better anti-tumor effect and a higher response rate can be expected in contrast to the IL-2 injection. In addition, the topical application allows the treatment of occult skin micro-metastases. As a consequence, a recurrence can be counteracted which is only partially or not at all the case in a surgical removal and an IL-2 injection. In addition, the topical application allows an effective melanoma prophylaxis, the therapy of melanoma precursors or, after the excision of a primary melanoma, the reduction of the risk of the primary melanoma formation or a melanoma recurrence, respectively.

Therefore, according to another embodiment of the pharmaceutical composition is provided in an application form which allows a topical application and is, for this reason, preferably selected from the group consisting of: ointment, cream, lotion, gel, paste, transdermal therapeutic system, foam, powder.

The active agents 6-PN and/or 8-PN can be provided in encapsulated form, for example in liposomes.

The composition according to the invention can be configured as a mono-preparation, where 6-PN and/or 8-PN are provided as (the) only active agent(s). According to a further embodiment of the invention the pharmaceutical composition comprises an additional active agent effective against melanomas and/or melanoma precursors and/or skin and/or mucosa metastases of any primary tumor.

This measure may take advantage of synergistic effects resulting from the cooperation of 6-PN and/or 8-PN and the additional active agent. This may also result in a simplification of the application by the patient who may apply only the pharmaceutical composition of the invention instead of two preparations.

In an embodiment of the invention the additional active agent is a necrosis inhibitor.

This measure has the advantage that necrotic processes and inflamations associated therewith are inhibited and any side effects of the composition according to the invention are further reduced.

According to another embodiment of the invention it is preferred if the necrosis inhibitor is IM-54.

By this measure such a necrosis inhibitor is used which is especially suited according to the findings of the inventors.

Another subject matter of the present invention relates to the use of prenylnaringenin, preferably 6- and/or 8-Prenylnaringenin, as an epigenetically active medicinal agent, further preferably for the prophylaxis and/or treatment of a melanoma and/or a melanoma precursor and/or a skin and/or mucosa metastasis of any primary tumor.

Another subject matter of the present invention relates to the use of prenylnaringenin, preferably 6- and/or 8-prenylnaringenin, as a histon deacetylase inhibitor (HDACi).

Another subject matter of the present invention is a method for the prophylaxis and/or treatment of a melanoma and/or a melanoma precursor and/or a skin and/or mucosa metastasis of any primary tumor in a living being, preferably a human being, comprising the following steps: (1) Providing the pharmaceutical composition according to the invention, and (2) application of the pharmaceutical composition onto the skin of the living being, preferably the melanoma and/or the melanoma precursor and/or the skin and/or mucosa metastasis.

The properties, features and advantages of the pharmaceutical composition according to the invention apply likewise to the uses according to the invention and the method according to the invention.

It goes without saying that the features mentioned before and those that will be explained hereinafter may not only be used in the particularly given combination but also in other combinations or alone without departing from the scope of the present invention.

The invention is now explained in further detail by means of embodiments from which further properties, features and advantages will result. The embodiments shall illustrate the invention, however do not restrict its scope. Reference is made to the enclosed figures:

### BRIEF DESCRIPTION OF THE DRAWINGS

FIG. 1: Chemical structures of 6-PN and 8-PN. The chemical structures of 6-prenylnaringenin (5,6-dihydroxy-2-(4-hydroxy-phenyl)-6-(3-methyl-but-2-enyl)-chroman-4-one) and 8-prenylnaringenin (5,7-Dihydroxy-2-(4-hydroxy-phenyl)-8-(3-methyl-but-2-enyl)-chroman-4-one) are shown.

FIG. 2: In silico docking analysis with 6-PN and 8-PN illustrates the inhibitory potential on human-derived HDACs of class I and II. (A) Results of the in silico docking analysis of 6-PN and 8-PN with crystal structures of HDAC2 (class I) and HDAC4 (class II). The analysis shows the predicted binding mode of 6-PN and 8-PN in the HDAC binding pocket. Both molecules are predicted to interact with the zinc ion as well as other residues of the catalytic center. Docking analyses of 6-PN and 8-PN in the individual HDAC binding pockets were performed using GOLD (version 4.1.2) and MOE (version 2009.10). (B) Overall HDAC inhibition in cellular extracts of the human cell line HeLa by increasing concentrations of 6-PN and 8-PN (5 μM, 10 μM, 20 μM, 50 μM and 100 μM). As reference inhibitor 100 μM SAHA was used. Every concentration was tested three times in triplicates. Shown are the mean±SD. (C) Specific fluometric profiling assay using recombinant human HDACs of classes I, II and IV. Specific inhibition values were generated for the treatment with 100 μM 6-PN and 8-PN. TSA was used as reference inhibitor (HDAC1, 2, 3, 6, 10, 11: 10 nM; HDAC4, 8, 9: 1 μM; HDAC5, 7:10 μM). The inhibition vales for every HDAC were yielded by one experiment performed in duplicates Shown are the mean±S.A.

FIG. 3: in silico docking analysis with 6-PN and 8-PN for HDAC2, HDAC4, HDAC7 and HDAC8. (A) Results of the in silico docking analysis of 6-PN and 8-PN with crystal structures of HDAC8 (class I) as well as HDAC7 (class II). The analysis shows the predicted binding mode of 6-PN and 8-PN into the different HDAC binding pockets. Both molecules are predicted to interact with the zinc ion as well as other residues of the catalytic center. (B) 2D ligand plot depiction of 6-PN and 8-PN along with interacting amino acids (circles) of the corresponding binding pocket. Shown are hydrophobic, polar, acidic and basic residues. Halo-like disks around the amino acids are calculated based on the reduction of solvent exposure by the ligand. The arrows represent backbone H-bond interactions or side chain H-bond interactions. The dotted lines represent metal contacts. The benzene rings with a “+” describe an arene-cation-interaction, 2 benzene rings and arene-arene-interaction. Areas with a colored background correspond to solvent exposed parts of the ligands. Docking analyses of 6-PN and 8-PN in the individual HDAC binding pockets were performed using GOLD (version 4.1.2) and MOE (version 2009.10).

FIG. 4: 6-PN and 8-PN induce a hyper acetylation in SK-MEL-28 melanoma cells. Western blot analysis of acetylated histon complex H3 in cellular lysates of BLM and SK-MEL-28 cells treated with 100 μM 6-PN or 8-PN for 1, 2, 4, 12 and 24 h, or with 100 μM SAHA (as reference HDACi) for 24 h. Equal protein loading was verified by vinculin staining (lower rows). At 24 hours (BLM) and 2 hours (SK-MEL-28) after the PN treatment a prominent up-regulation of ac-H3 is detectable. The protein expression levels were estimated performing a densitometric analysis.

FIG. 5: real time cell monitoring of BLM and SK-MEL-28 melanoma cells treated with 6-PN and 8-PN. (A) BML and SK-MEL-28 cells were treated 24 hours after seeding with different concentrations of 6-PN and 8-PN (20 μM, 50 μM and 100 μM) over a 104 hour time period. The cellular impedance was measured over the entire observation time using the xCELLigence™ SP system and is depicted by the cell index (CI). All CI-values were normalized shortly before the treatment started. Displayed are normalized CI values every four hours. As a positive control, Triton X-100 0.1% was used to induce cell death. Shown are the mean±SD of three independent experiments, each performed in triplicates (B, C). MUH proliferation assay of BLM and SK-MEL-28 melanoma cells treated with 6-PN and 8-PN (0.1 μM to 100 μM), TSA and SAHA (both at 100 μM), with and without the addition of the necrosis inhibitor IM-54 (5 μM) demonstrates a concentration-dependent reduction of the cell proliferation >90% in both cell lines by the PNs that is partly inhibited by IM-54. Shown are the mean±SD; the experiments were performed in quadruplicates. (D) The MUH proliferation assay was repeated unter the same treatment conditions on primary human fibroblasts (FF868) and melanocytes (HEM1).

FIG. 6: 6-PN and 8-PN inhibit the growth of MCF-7 and HT-29. (A, B) MUH proliferation assay of HT-29 colon cancer cells and MCF-7 breast cancel cells treated with increasing concentrations of 6-PN and 8-PN (0.1 μM to 100 μM) for 48 hours. Shown are the mean±S.A.; the experiments were performed in quadruplicates.

FIG. 7: 100 μM 6-PN and 100 μM 8-PN do not induce cleaved caspase-3 in SK-MEL-28 melanoma cells. Western blot analysis of SK-MEL-28 melanoma cells treated with 100 μM of 6-PN, 8-PN, SAHA or TSA. EtOH and DMSO were applied as controls. Although a band for caspase-3 (25 kD) is clearly visible for all treatments, no additional band for cleaved-caspase-3 (expected size: 17 kDa) was found upon any of the treatments.

FIG. 8: 6-PN and 8-PN decrease expression of pS6p via down regulation of pERK/pP90. Western blot analysis of SK-MEL-28 melanoma cells 1 hour, 2 hours, 4 hours, 12 hours, and 24 hours after treatment with 100 μM 6-PN or 8-PN. 6-PN and 8-PN induced down regulation of pS6p accompanied by a decrease of pERK and pP90, respectively. 6-PN and 8-PN induced time dependent increase of p70s6-kinase.

FIG. 9: 100 μM 6-PN and 100 μM 8-PN do not induce apoptosis in SK-MEL-28 melanoma cells. Apoptosis assay (35 proteins) performed on lysates of SK-MEL-28 melanoma cells 4 hours after treatment with 100 μM of 6-PN or 8-PN. Protein expression levels were estimated performing a densitometric analysis. Neither 6-PN nor 8-PN yielded a significant change of expression of the analyzed proteins involved in apoptosis.

FIG. 10: 100 μM 6-PN and 100 μM 8-PN do not induce apoptosis in SK-MEL-28 melanoma cells. (A) Cell cycle FACS analyses of PI stained BLM and SK-MEL-28 melanoma cells. 100 μM of 8-PN-induced a G2 arrest in both cells. (B) FACS analyses of BLM and SK-MEL-28 melanoma cells treated with 100 μM 6-PN or 8-PN and stained for annexin V/PI; staurosporin was used as control for apoptosis induction and DMSO as vehicle control. PN-treatment did not increase the number of annexin V positive (apoptotic) cells. (C) BLM and SK-MEL-28 melanoma cells were treated with 100 μM of PNs; TSA, SAHA and EMSO were used as controls. Western Blot was performed using an antibody against LC3 and showed a shift from LC3-I to LC3-III (second, lower band) after treatment with the PNs after 12 and 24 hours.

FIG. 11: 6-PN and 8-PN reduce proliferation and invasion of BLM and LOXIMVI melanoma cells in human epidermal skin reconstructs and induce little embryo toxicity. (A) BLM melanoma cells and keratinocytes were seeded on top of a collagen matrix populated by fibroblasts. After 16 days a keratinizing stratified epithelium with a rudimentary corium is formed. Untreated, MIB1 positive (proliferating) control BLM cells form a large tumor in the epidermal part of the reconstruct and invade the corium (upper panel. For untreated BLM cells 103±15 MIB1 positive and 39±13 invasive cells were counted per high-power filed (40× magnification). 100 μM 6-PN treatment yielded a significantly decreased amount of MIB1 positive BLM cells (27±6) with less invasion (9±4; middle panel; p<0.01, Student's t-test). 100 μM 8-PN further reduced proliferation (16±4) and Invasion (6±2) of BLM cells compared to the untreated cells (lower panel; p<0.01, Student's t-test). Experiments were performed in triplicates. (B) Similar results were obtained for LOXIMVI melanoma cells under the same treatment conditions. Only nests of dead, MIB1 negative LOXIMVI cells were detected after treatment with the PNs. (C) Chicken embryos stage 13HH (corresponding to 6 human gestational weeks) were exposed to DMSO as control, 100 μM 6-PN or 100 μM 8-PN to determine LD50 values. Survival rates after 24 hours, 48 hours, and 72 hours are depicted in a Kaplan-Meier plot.

### DESCRIPTION OF PREFERRED EMBODIMENTS

### 1. Material and Methods

1.1 Synthesis of 6-prenylnaringenin and 8-prenylnaringenin (PN)

8-Prenylnaringenin and 6-prenylnaringenin were synthesized according to Gester et al. (2001), An efficient synthesis of the potent phytoestrogens 8-prenylnaringenin and 6-(1,1-dimethylallyl)naringenin by europium (III)-catalyzed Claisen rearrangement, Tetrahedron 57, 1015-1018 and Tischer and Metz (2007), Selective C-6 prenylation of flavonoids via europium(III)-catalyzed Claisen rearrangement and cross-metathesis, Advanced Synthesis & Catalysis 349, 147-151, synthetized. The content of both before-mentioned publications is incorporated herein by reference.

### 1.2 Cell Culture

The inventors have tested 19 different tumor cell lines in total, including five melanoma cell lines, two liver cancer cell lines, three breast cancer cell lines, three colon cancer cell lines, two prostate cancer cell lines, two lung cancer cell lines and to renal cancer cell lines.

TABLE 1
Tested tumor cell lines

| Tumor entity | Cell line |
|---|---|
| Malignant melanoma | LOXIMVI, SK-MEL-28, SK-MEL-5, |
| | BML, Malme-3M |
| Liver cancer | HepG2, Hep3B |
| Breast cancer | MCF7, BT-549, MDA-MB-231 |
| Colon cancer | HT29, HCT-116, COLO-205 |
| Prostate cancer | PC-3, DU-145 |
| Lung cancer | A549, NCI-H522 |
| Renocancer | UO31, TK10 |

As example in the following metastatic melanoma cell lines SK-MEL-28, LOXIMVI and BLM, the colon cancer cells HT29 and the breast cancer cells MCF-7 are described. They were cultured in RPMI1640 medium supplemented with 10% fetal bovine serum (FBS), 1% Penicillin, and 1% Streptomycin. All cell cultures were maintained at 37° C. in a 95% air/5% CO₂ atmosphere at 100% humidity.

### 1.3 In Silico Docking Analyses

The docking analyses were performed with human HDAC2, 4, 7, and 8 with 6-PN, 8-PN, and the two reference HDACi compounds suberoyl anilide hydroxamic acid (SAHA) and trichostatin A (TSA).

### 1.4 HDAC Inhibitor Screening Assay

The determination of the HDACi activity was performed with the HDAC assay kit as described by the manufacturer (Active Motif, La Hulpe, Belgium), using 6-PN and 8-PN at rising concentrations (5 μM, 10 μM, 20 μM, 50 μM and 100 μM).

### 1.5 HDAC Inhibition Profiling

The human HDAC profiling assay was performed on the basis of the Fluor de Lys™ technology by Scottish Biomedical, Glasgow, United Kingdom. The percentage inhibition values of 100 μM of 6-PN and 100 μM of 8-PN against the human HDAC enzymes HDAC1, HDAC2, HDAC3, HDAC4, HDAC5, HDAC6, HDAC7, HDAC8, HDAC9, HDAC10, and HDAC11 were determined. All assays were performed in 1% DMSO (final concentration).

### 1.6 Immuno-Blotting

The following antibodies were used: Anti-Vinculin and Anti-Actin (1:5000, Sigma-Aldrich), Anti-Acetyl-Histon H3 (1:5000, Millipore, Billerica, USA), Anti-Caspase-3 (1:1000), Anti-AKT, Anti-pAKT, Anti-ERK, Anti-pERK, Anti-pE90, Anti-p70S6 Kinase, and Anti-pS6 protein (1:1000), all from Cell Signaling, Frankfurt, Germany), Anti-LC3 (1:5000, Sigma-Aldrich).

### 1.7 Proliferation Assay

The cells were seeded in triplicates in 96-well plates at a density of 2,500 cells per well in 50 μl medium (5×10⁴ cells per milliliter). After 24 hours, the medium was replaced by medium containing 6-PN, 8-PN (dissolved in DMSO), TSA (dissolved in EtOH) or SAHA (dissolved in DMSO) at the concentrations to be tested with or without the addition of the necrosis inhibitor IM-54 at 5 μM (Calbiochem, Darmstadt, Germany). Cells treated with culture medium without or with DMSO served as controls. The assay was started following an incubation of 24 hours. The medium was discarded, each well was washed two times with PBS (without Ca⁺⁺ and Mg⁺⁺) and 100 μl of a solution containing 100 mg of 4-methyl umbellipheryl heptanoate per ml PBS was added. Plates were incubated at 37° C. for 1 hour and measured in a Fluoroskan II (Lab Systems, Helsinki, Finland) with a λem of 355 nm and λex of 460 nm. The intensity of fluorescence indicates the number of viable cells in the wells.

### 1.8 Real Time Cell Monitoring Assay

The human melanoma cell lines BLM and SK-MEL-28 (2.5×10³ cells/well) were seeded in 96 well plates. The cells were treated after 24 hours with different concentrations of 6-PN or 8-PN (20 μM, 50 μM and 100 μM) and monitored by measurements of electrical impedance in 15 minutes intervals for an overall time span of in total 104 hours using the xCELLigencer® SP Systems (Roche Applied Science). The cell index values were calculated using the RTCA software (1.0.0.0805). All curves were normalized at the beginning of the treatment period.

### 1.9 Apoptosis Assay

Testing of apoptosis induction was performed with the human Proteom-Profiler-Apoptosis-Antibody-Array-Kit (R & D Systems, Wiesbaden, Germany) as described by the manufacturer after treatment of SK-MEL-28 melanoma cells with 100 μM 6-PN or 100 μM 8-PN for 4 hours.

### 1.10 Organotypic Epidermal Skin Reconstructs

Organtypic cultures of human skin and melanoma (using BLM and LOXIMVI melanoma cells) were generated as previously described by Meier et al. (2000), Human melanoma progression in skin reconstructs: biological significance of bFGF, Am. J. Pathol. 156, 193-200.

### 1.11 Embryo Toxicity Testing (Determination of LD₅₀)

Fertilized eggs of leghorn chickens (Gallus gallus domesticus) were incubated at 38° C. in a temperature-controlled, humidified brooder. The upper most spot of the egg shell and thus indirectly the blastoderm, which is always oriented towards the top part of the egg, was marked on each egg with a permanent marker. For embryo toxicity testing, the eggs were prepared after approximately 48 to 52 hours of incubation (equal to stage 12 to 13), which corresponds to approximately 6 human gestational weeks. Before fenestration, a small hole was pierced into the lateral edge of the eggs and 2 ml of albumen were withdrawn with a syringe from the lower level of the blastoderm. Subsequently, the egg was prepared for fenestration by using a hack saw to generate a rectangular predetermined breaking point on the shell around the previously marked spot (about 5×25 mm in size). The predetermined “window” was opened by removal of the egg shell with bent forceps. At this stage, the embryo is already visible on top of the blastoderm. 100 μM of 6-PN or 8-PN (dissolved in 50% ethanol and 50% PBS, total volume: 500 μl) was applied on the top of the blastoderm (2 embryos per treatment group; the experiment was performed in triplicates; total n=6 per group). As control, the same number of embryos were treated with the same volumes of ethanol and PBS without the addition of 6-PN or 8-PN to exclude possible toxic effects of ethanol. The eggs were then sealed with adhesive tape and replaced into the incubator. The viability of the embryos was verified and documented 24, 48 and 72 hours after the application of the substances.

### 1.12 Statistical Analyses

Statistical analyses for different assays were performed with the One-Way ANOVA Dunnett's Multiple Comparison Test using GraphPad Prism Version 4.00 (GraphPad Software, San Diego, Calif., USA). According to this analysis a value of p<0.01 was defined as statistically significant.

### 2. Results

2.1 in Silico Docking Analyses Predict Binding of 6-PN and 8-PN into Human HDAC Enzymes.

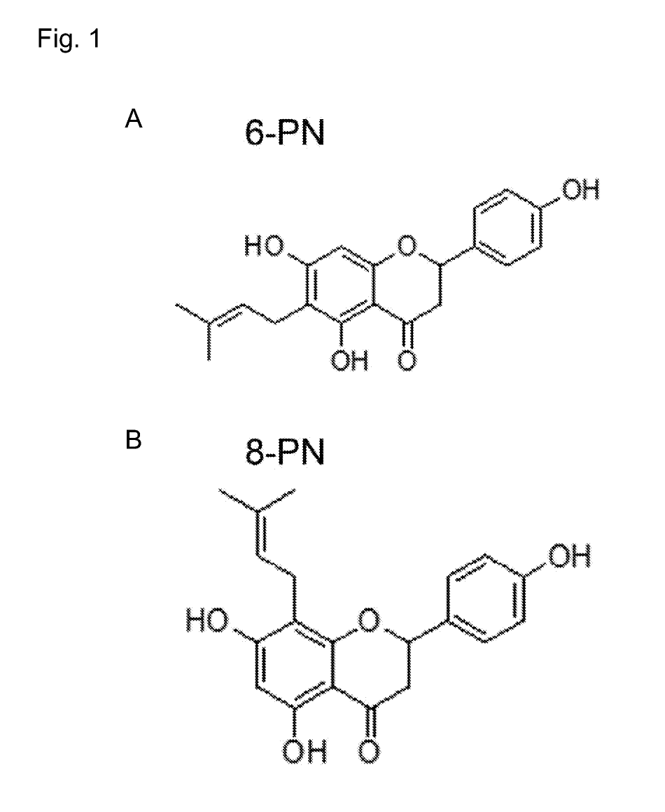

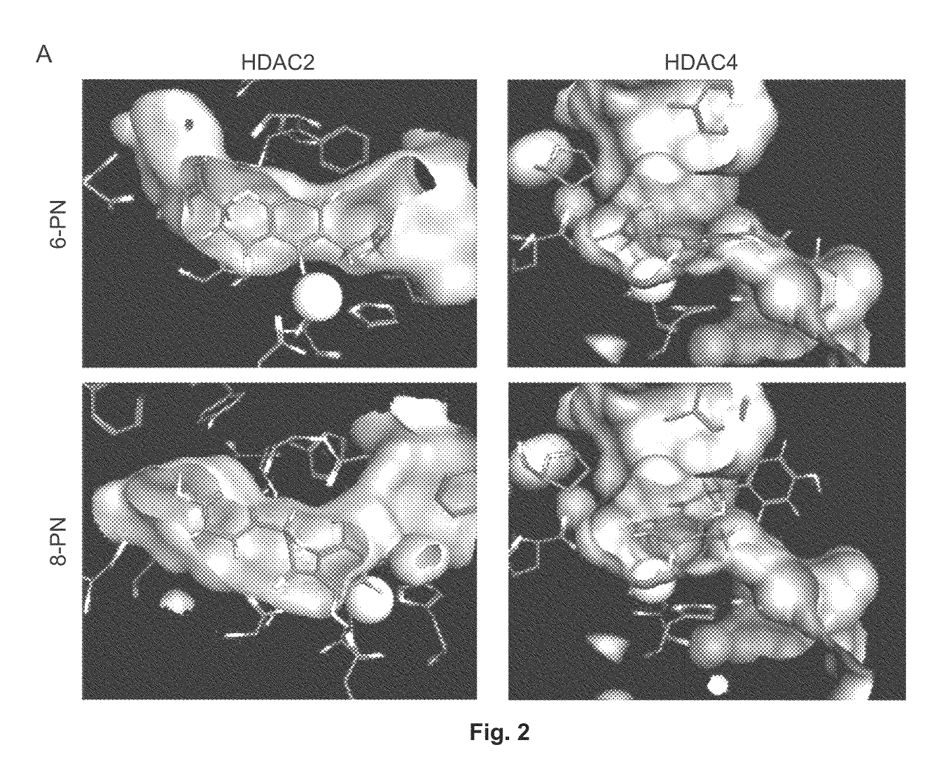

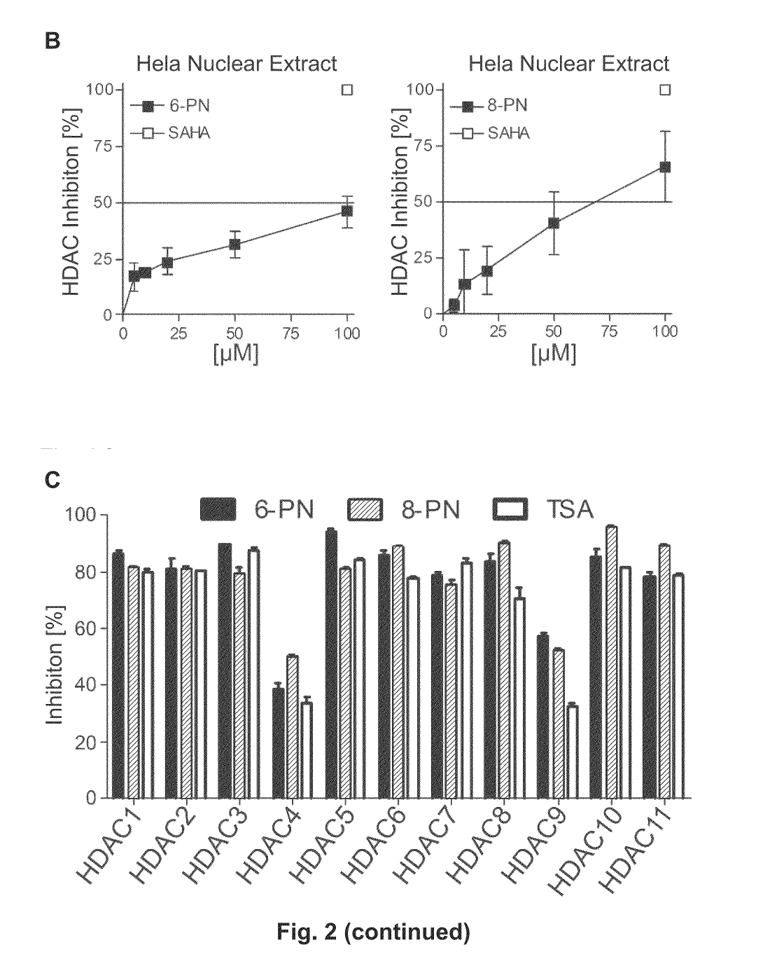

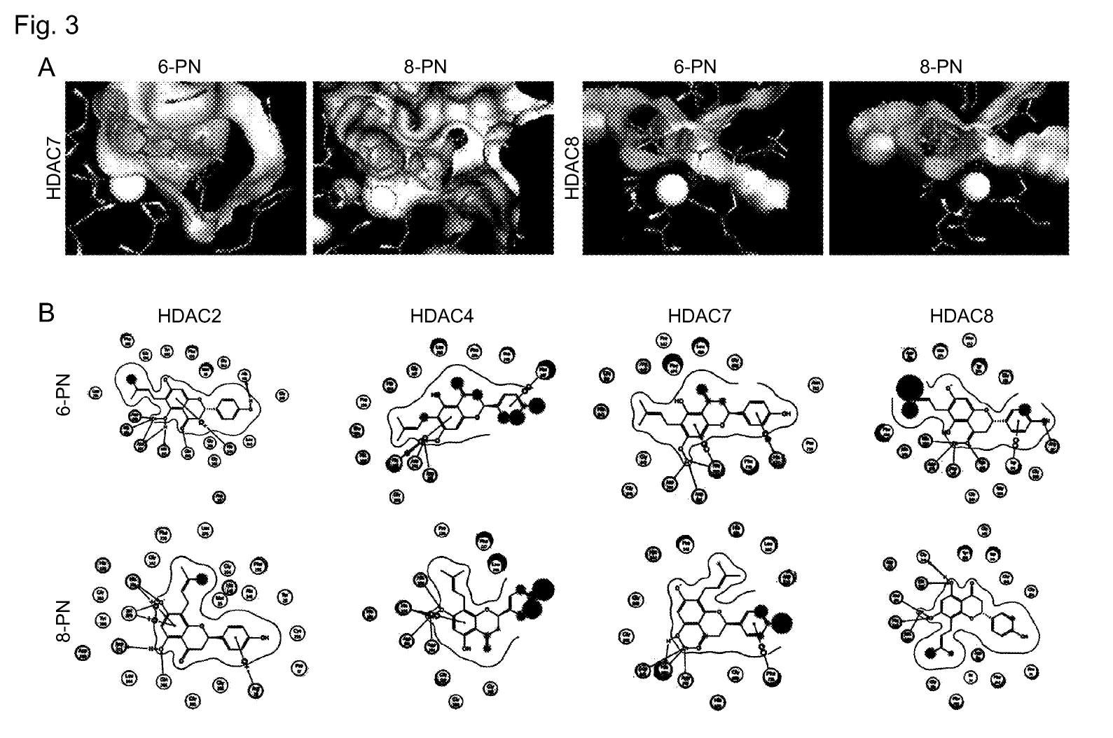

To identify potential HDACi activity of 6-PN and 8-PN (FIG. 1), first in silico docking analyses were performed with human HDAC class I members HDAC2 and HDAC8 as well as the class II members HDAC4 and HDAC7 (FIG. 2, 3). Important features for a HDACi are the structural property to fit into the binding pocket of HDAC enzymes and the capacity to interact with key residues like the zinc ion in the catalytic center. According to the predicted interactions of the docking analyses 6-PN and 8-PN theoretically fulfill both requirements concerning HDAC2, HDAC4 (FIG. 2A) or HDAC7 and HDAC8, respectively (FIG. 3). However, it was observed that no consistent binding mode was obtained for 6-PN and 8-PN across the different HDAC enzymes, i.e. different parts of 6-PN and 8-PN interact with the zinc ion. This renders the docking analysis of 6-PN and 8-PN difficult to interpret. The established HDACi tricholstatin A (TSA; not clinically approved due to toxicity) and suberoyl anilide hydroxamic acid (SAHA; clinically approved for cancer therapy) were also analyzed as reference inhibitors. For comparison of the docking results the scoring function GoldScore was used. Interestingly, 6-PN and 8-PN yielded higher GoldScores than TSA or SAHA for HDAC4, HDAC7 and HDAC8, and GoldScores comparable to (6-PN) or lower (8-PN) than TSA or SAHA for HDAC2 with the above-mentioned restriction.

TABLE 2
GoldScores for 6-PN and 8-PN. Calculated GoldScores for 6-PN
and 8-PN on the basis of the corresponding docking analysis with
HDAC2, 4, 7 and 8. The docking and following analyses were
made by using GOLD (version 4.1.2).

| | HDAC2 | HDAC4 | HDAC7 | HDAC8 |
|---|---|---|---|---|
| 6-PN | 62.8 | 66.5 | 56.9 | 59.5 |
| 8-PN | 37 | 57.6 | 56.7 | 64.8 |

Based on the in silico data 6-PN and 8-PN seem to exhibit inhibitory activity against HDAC enzymes of class I and II comparable to standard HDACi like TSA and SAHA.

### 2.2 In Vitro Screening Confirms a Pronounced Pan-HDACi Activity of 6-PN and 8-PN.

To further substantiate the predicted HDACi activity in vitro, 6-PN and 8-PN were screened in a HDACi assay. Employing standardized nuclear extract of HeLa cells as human HDAC enzyme source, 6-PN and 8-PN showed a dose dependent HDAC inhibitory activity (FIG. 2B). Low concentrations of 6-PN (5 μM) and 8-PN (10 μM) already showed inhibitory effects, and 100 μM resulted in an inhibition rate of ≧50% (FIG. 2B). 100 μM SAHA was used as control HDACi. In conclusion, 6-PN and 8-PN displayed a substantial HDACi activity on human HDAC enzymes in vitro.

To prove the in silico and in vitro results and to apportion the inhibitory activity of 6-PN and 8-PN, a profiling analysis with all known human HDAC enzymes of class I, II and IV was performed (FIG. 2C). On the basis of the HDACi screening assay and the resulted inhibition values, 100 μM 6-PN, 8-PN and TSA (as a reference HDACi; in the optimized concentrations of 0.01 μM, 1 μM and 10 μM) were chosen to test all conserved eleven HDAC enzymes and to ensure adequate inhibitor concentration. In line with the docking results and the screening assay, 6-PN as well as 8-PN strongly inhibited HDAC activity on all tested human HDACs (FIG. 2C). According to the specific HDAC inhibition values for HDAC1-HDAC11, both prenylflavonoids can be considered as pan-HDACi.

2.3 Hyperacetylation of Histon H3 after 6-PN and 8-PN Treatment in BLM and SK-MEL-8-Melanoma Cells

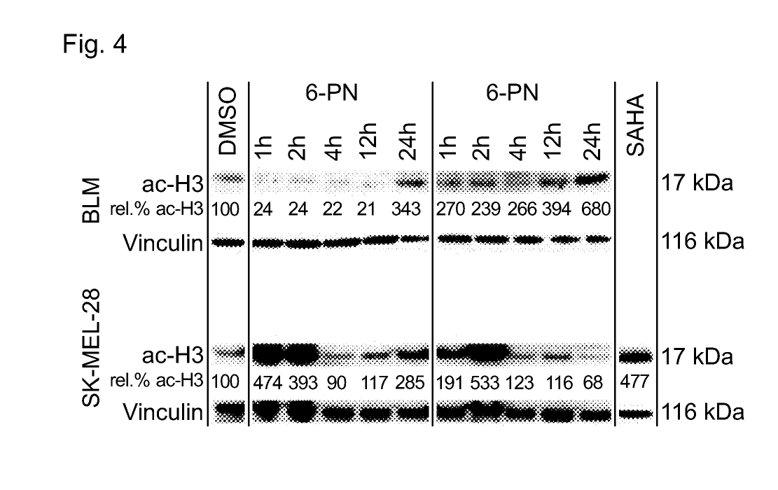

An incubation of cells with HDACi induces generally a hyperacetylation of histon proteins. Therefore, the acetylation status of the histon protein H3 after treatment with 6-PN or 8-PN was investigated. Corresponding to the HDAC screening results, a direct effect was monitored. Western blot analyses revealed a massive increase of histon complex H3 acetylation in BLM melanoma cells 24 hours after treatment with 100 μM of 6-PN or 8-PN, and in SK-MEL-28 melanoma cells already two hours after the treatment with 100 μM of 6-PN or 8-PN, when compared to DMSO treated control cells (FIG. 4). SAHA was used as control HDACi (at 100 μM, 24 hours treatment).

### 2.4 Apoptosis-Independent Antiproliferative Effects of 6-PN and 8-PN on Human Melanoma, Colon and Breast Cancer Cells.

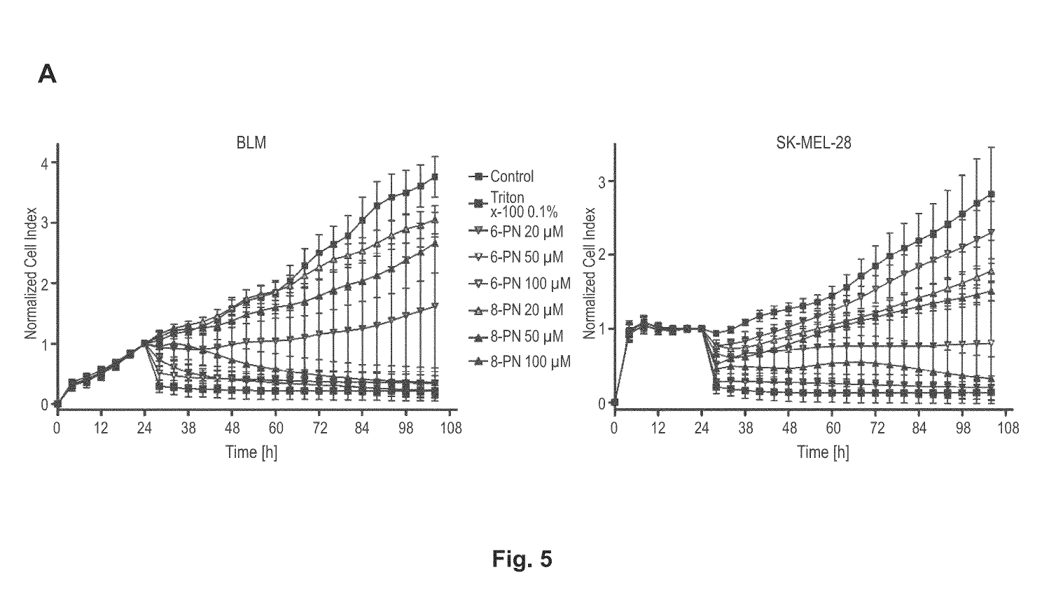

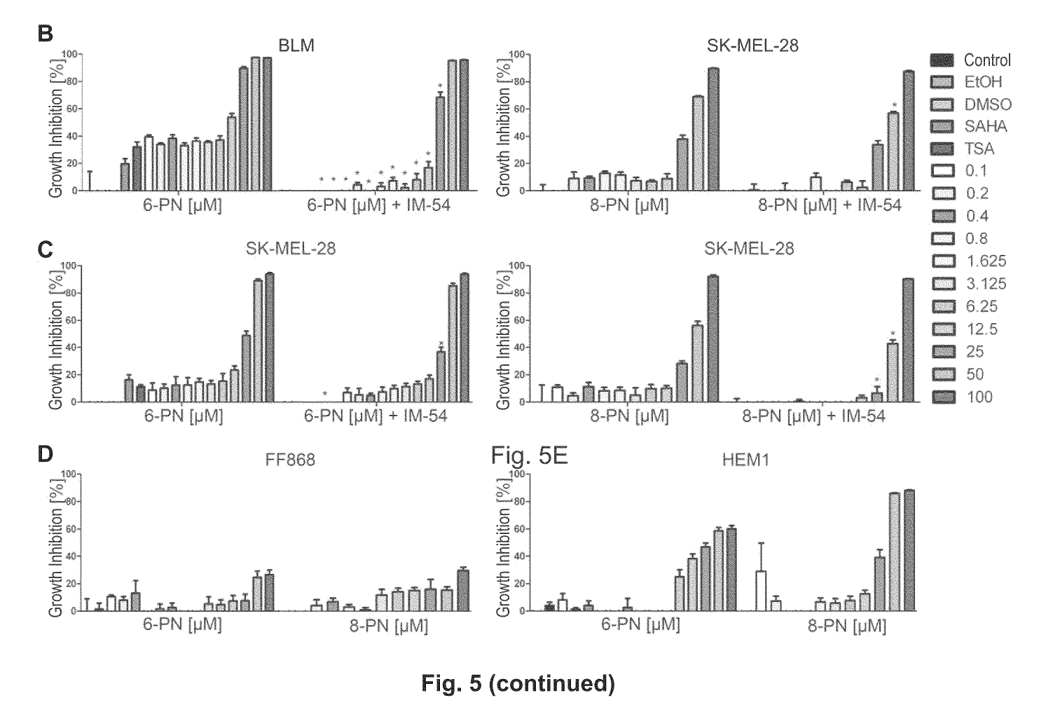

Due to the new found HDACi activity of 6-PN as well as 8-PN and the observed hyper acetylation of histon protein H3 in human melanoma cells, the antiproliferative effects on cancer cells were tested. The human metastatic melanoma cell lines BLM and SK-MEL-28 were treated once with rising concentrations of 6-PN or 8-PN (20 μM, 50 μM and 100 μM) and monitored continuously for an overall time span of 104 hours using a real time monitoring assay (FIG. 5A). The cell's impedance, depicted as cell index (CI), was measured reflecting changes of the cellular status. Hence, a rise of the CI generally displays cellular growth, whereas a decrease indicates pertubations of cell growth or viability. The normalized CI of BLM and SK-MEL-28 cells treated with 6-PN and 8-PN descended over time in comparison to the control for every concentration tested. Especially an incubation with 50 μM and 100 μM of 6-PN as well as 8-PN showed a pronounced and rapid change of the measured CI. The early HDACi-mediated hyperacetylation of histon H3 detected by the western blot analysis at 100 μM for both PNs in SK-MEL-28 cells was in line with the rapid decline of the normalized CI already 4 hours after treatment in contrast to the control. Likewise, in BLM cells hyperacetylation of histon H3 upon treatment with the PNs corresponds to a delayed decline of the CI.

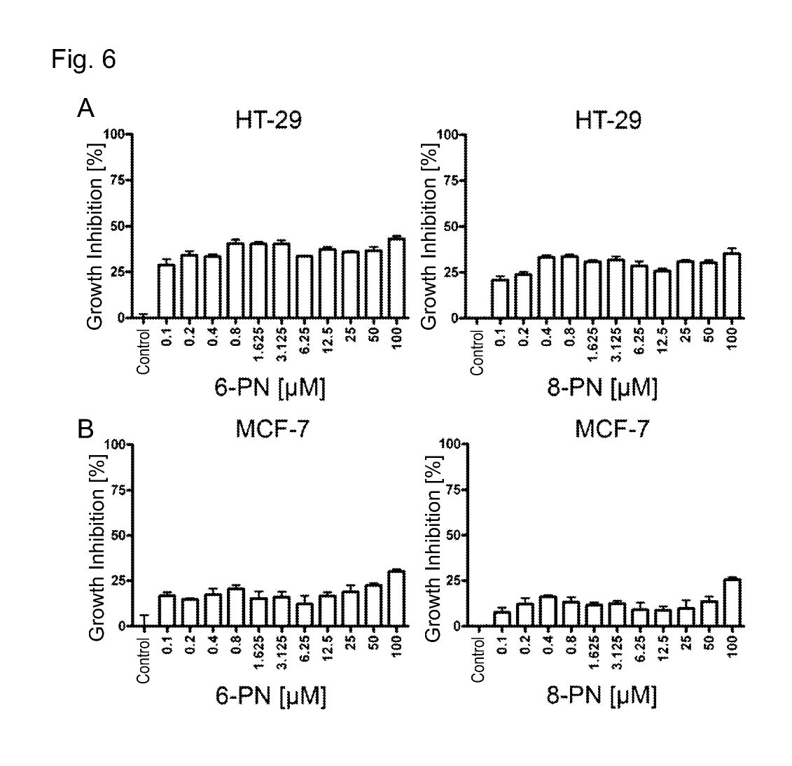

In order to substantiate the antiproliferative effects of 6-PN and 8-PN on BLM and SK-MEL-28 melanoma cells, proliferation assays were performed (FIG. 5B, C). According to the real time monitoring assays already 20 μM of 6-PN or 8-PN induced a distinct decline within 48 hours (FIG. 5A). Therefore on the one hand also low concentrations were included in this assay and on the other hand the cell lines were treated for 48 hours. In concordance with previous results, concentrations of 50 μM and 100 μM of 6-PN as well as 8-PN showed a pronounced antiproliferative effect. 6-PN reduced cell proliferation up to 95% and 8-PN up to 90% in BLM and SK-MEL-28 cells (FIG. 5B, C). Further, also treatment of other tumor entities like HT-20 colon cancer and MCF-7 breast cancer cells with rising concentrations of 6-PN or 8-PN induced a severe reduction of cell proliferation (FIG. 6).

To further evaluate the anti cancer properties of 6-PN and 8-PN on metastatic melanoma cells the proliferation assays were repeated with the addition of the necrosis inhibitor IM-54 to analyze the role of necrosis induction upon the treatment with PN. The addition of 5 μM IM-54 decreased cell death rates at higher PN concentrations in both BLM and SK-MEL-28 cell lines by up to 35% (FIG. 5B, C). Higher doses of IM-54 (10 μM) did not show an additional effect (not shown).

Next it was asked whether the PNs also influence the proliferation of benign cells. To this end, human fibroblasts (FF) and foreskin melanocytes (HEM1) were treated with the same concentrations of 6-PN and 8-PN, and proliferation was assessed using the proliferation assay as described above. While fibroblast proliferation was hardly affected by the PN treatment (FIG. 5D), the melanocyte proliferation was markedly decreased at higher concentrations of PNs (FIG. 5E). For TSA and SAHA no reduced proliferation of the fibroblasts or melanocytes could be observed.

Taken together, 6-PN and 8-PN effectively reduce cell proliferation in all tested cancer cells (cf. Tab. 1); especially melanoma cells were highly susceptible towards the treatment with the two prenylflavonoids. IM-54 was able to partially block PN-induced cell death in BLM and SK-MEL-28 cells, suggesting that the antiproliferated and cytotoxic cascade initiated by PN eventually might lead to the induction of necrosis in a fraction of the melanoma cells.

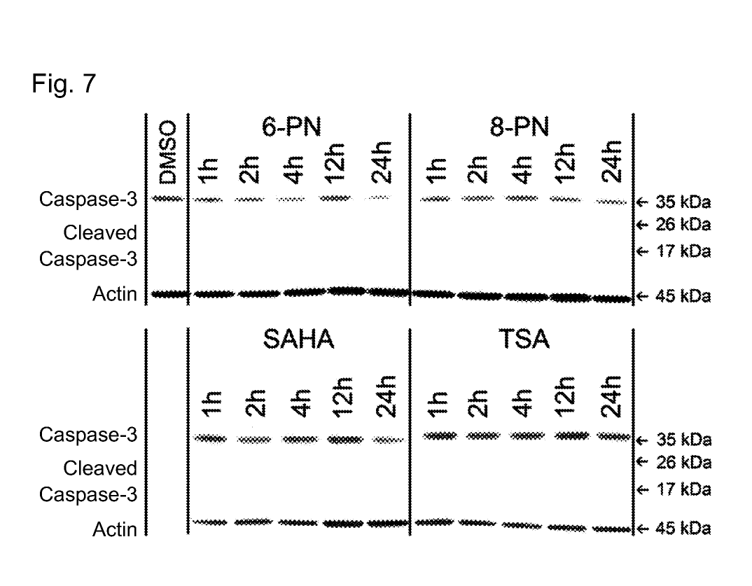

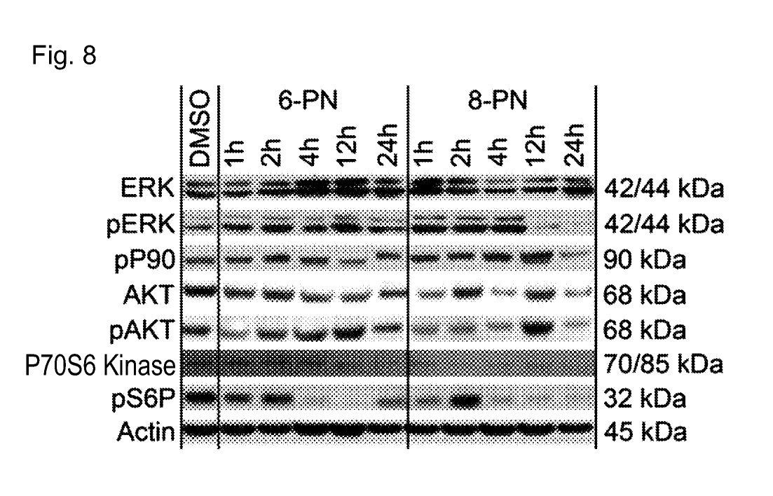

To further analyze the type of cell death induced by 6-PN and 8-PN, western blot analyses of SK-MEL-28 melanoma cells were performed 1, 2, 4, 12 and 24 hours after the treatment with 100 μM 6-PN and 8-PN. For both PNs decreased protein levels of caspase-3 (proapoptotic) and Bcl-xl (anti-apoptotic) were detected (not shown). However, no additional band for the cleaved caspase-3 for 6-PN, 8-PN or any of the applied reference HDACi (TSA, SAHA) or the vehicle control (DMSO) in SK-MEL-28 cells was detected (FIG. 7). Upon PN treatment an inconsistent expression of oncogenic AKT and phospho-AKT (pAKT) was detected. On the other hand, phospho-S6 protein (pS6P, a downstream target of mTOR) was strongly down regulated by 6-PN or 8-PN after 4 hours, 12 hours, and 24 hours (FIG. 8). Thus, there was an obvious discrepancy between the expression of pAKT and pS6P. Therefore it was first also looked for the expression of P70S6 kinase and its phosphorylated form. 6-PN and 8-PN down-regulated the bands for the P70S6 kinase in a time-dependent manner (FIG. 8). Interestingly, its phosphorylated form (pP70S6 kinase) was not detectable, even in the control group (not shown). Therefore it was next asked whether the S6P could be phosphorylated by a different mechanism. In this respect, activated ERK can phosphorylate the p90 ribosomal S6 kinase (pP90). pP90 also phosphorylates the S6P (in addition to the P70S6 kinase). Since the pP70S6 kinase was absent in the used cells (SK-MEL-28 and BLM), it was hypothesized that the phosphorylation of the S6P was conducted via the pERK/pP90 pathway, independent from the P70S6 signalling. This hypothesis was supported by the parallel down-regulation of the bands for pP90 (Thr359/Ser363) and pS6P 12 hours after the treatment with 6-PN, and the reappearance of both bands after 24 hours (FIG. 8). In the 8-PN treatment group a strong down regulation of pERK was detected after 12 hours and 24 hours and an additional decrease of pP90 protein, which was accompanied by the parallel down regulation of pS6P. The western blot experiments were repeated with BLM cells under the same treatment conditions and yielded similar results (not shown). Taken together, these results suggest that the phosphorylation of the S6P was conducted via the down-regulation of the pERK/pP90 pathway by the PNs, independent from PI3K/AKT/P70S6 signalling.

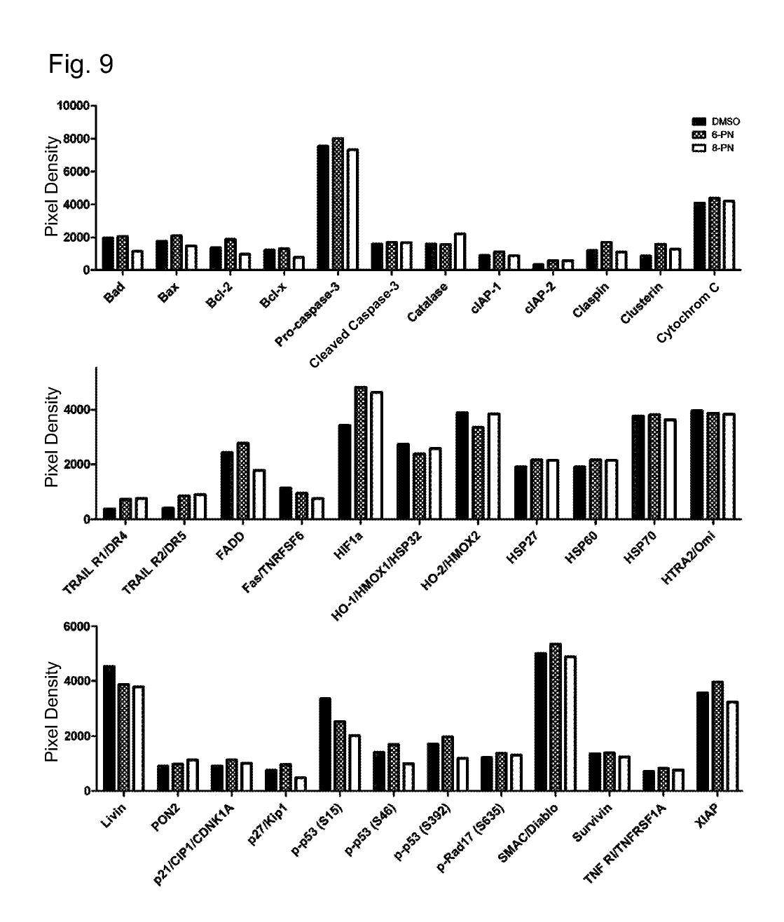

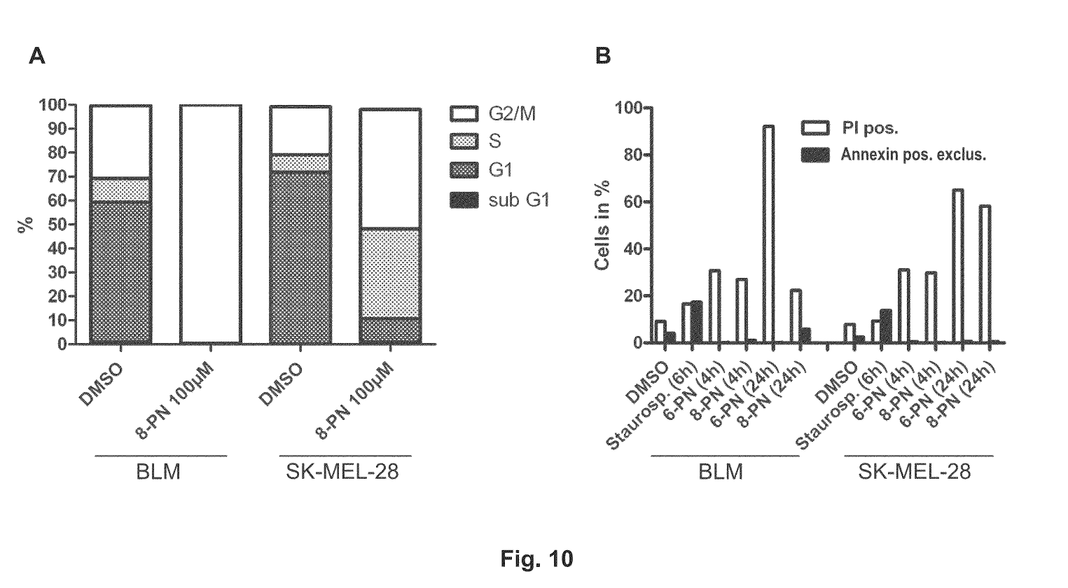

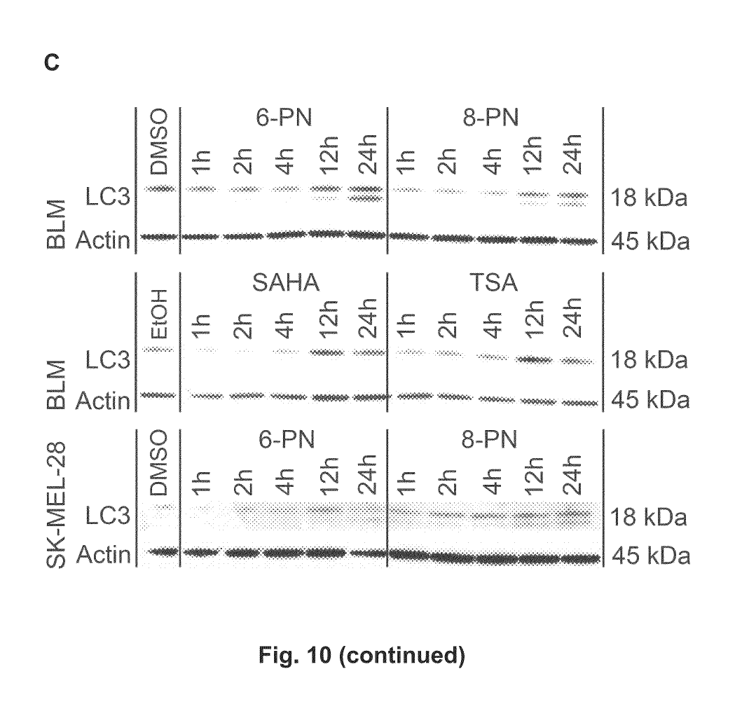

To further analyze the possibility of apoptosis induction, an extensive apoptosis assay was performed on SK-MEL-28 melanoma cells 4 hours after treatment with 100 μM 6-PN or 8-PN (FIG. 9). The protein expression levels were estimated performing a densitometric analysis and confirm that the decrease in the proliferation of melanoma tumor cells induced by 6-PN or 8-PN was not apoptosis related (FIG. 9). This was verified by FACS cell cycle analyses to screen for a possible increase of the sub-G1 fraction of cells (apoptotic cells) or for the induction of a G2 arrest (inhibition of proliferation). Indeed, the FACS analysis showed an induction of G2 arrest in BLM and SK-MEL-28 cells after 48 hours after the treatment with 100 μM of 8-PN when compared to the DMSO treated control cells (BLM: 99.6% vs. 30.4%; SK-MEL-28: 49.8% vs. 20.1% of cells in the G2 phase, respectively); the sub-G1 fraction (apoptotic cells) remained unaffected by 8-PN (FIG. 10A). To further differentiate between the induction of apoptosis and/or necrosis an PI/Annexin V staining of SK-MEL-28 and BLM cells was performed 4 hours and 24 hours after the treatment with 6-PN or 8-PN; 10 μM staurosporin (6 hours treatment) was used as positive control for the induction of apoptosis; DMSO treatment was used as vehicle control. The cells were then analyzed by FACS. In line with the results above, neither 6-PN nor 8-PN increased the number of annexin V positive (apoptotic) cells at 4 hours or 24 hours after treatment; staurosporin increased the number of annexin V positive cells (BLM: DMSO control: 4.1%; staurosporin: 17.4%; 6-PN 4 hours and 24 hours: 0.2% and 0.2%; 8-PN 4 hours and 24 hours: 1.2% and 5.9%; SK-MEL-28: DMSO control: 2.5%; staurosporin: 13.9%; 6-PN 4 hours and 24 hours: 0.5% and 0.6%; 8-PN 4 hours and 24 hours: 0.2% and 0.5%; (FIG. 10B). In contrast, the number of PI positive (nectrotic) cells strongly increased only after the treatment with the PNs (BLM: DMSO control: 9.1%; staurosporin: 16.6%; 6-PN 4 hours and 24 hours: 30.8% and 92.1%; 8-PN 4 hours and 24 hours: 27% and 22.4%; SK-MEL-28: DMSO control: 7.9%; staurosporin: 9.3%; 6-PN 4 hours and 24 hours: 31.1% and 65%; 8-PN 4 hours and 24 hours: 29.9% and 58.2% (FIG. 10B). Taken together, the western blot and FACS results clearly demonstrate that the 6-PN- and 8-PN-induced cytotoxicity on melanoma cells is not conducted by apoptosis induction, but rather by necrosis.

### 2.5 6-PN and 8-PN Induced Autophagy in BLM and SK-MEL-28 Melanoma Cells

Since it has been speculated that the formation of intracytoplasmic vacuoles in pancreatic or breast cancer cells by 6-PN and 8-PN observed in pervious studies might represent an induction of autophagy, this issue was further investigated on a molecular level. BLM and SK-MEL-28 melanoma cells were treated with 100 μM of 6-PN, 8-PN, SAHA or TSA (and with DMSO as control), and the treated cells were lysed for protein extraction after 1 hour, 2 hours, 4 hours, 12 hours and 24 hours. As indicator for the induction of autophagy, the shift from LC3 (LC3-I) to the autophagic, vesicle-associated form (LC3-II), was used. In both melanoma cells after 12-24 hours only 6-PN and 8-PN had clearly induced the shift from LC3-I to LC3-II (FIG. 10C).

### 2.6 6-PN and 8-PN Inhibit Proliferation and Invasion of BLM and LOXIMVI Melanoma Cells in Organotypic Human Epidermal Skin Reconstructs

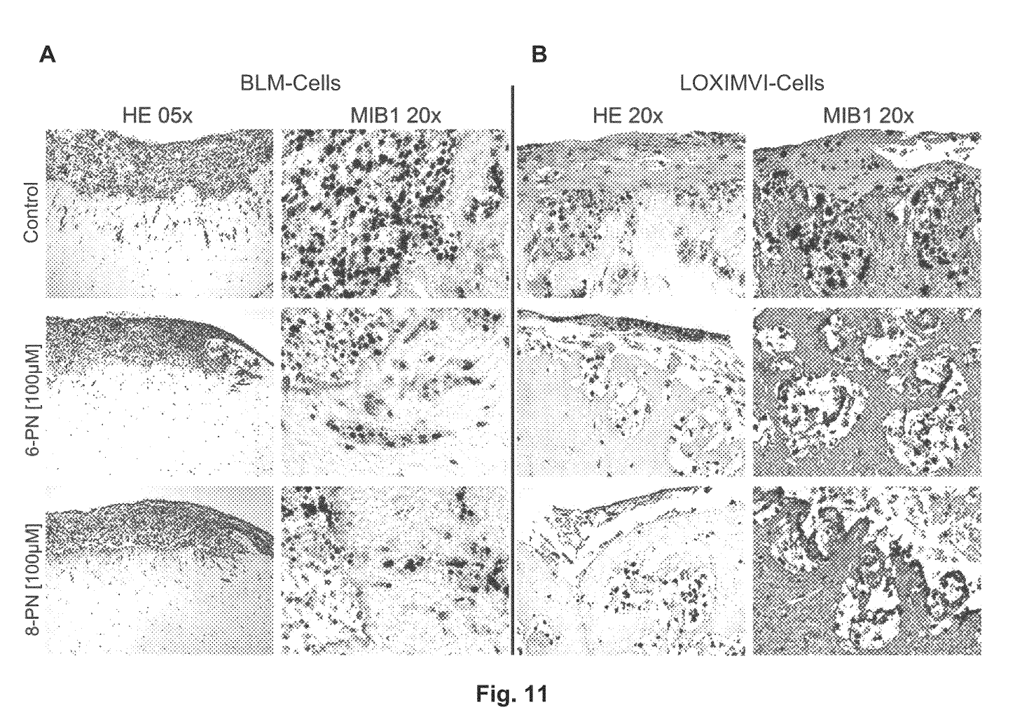

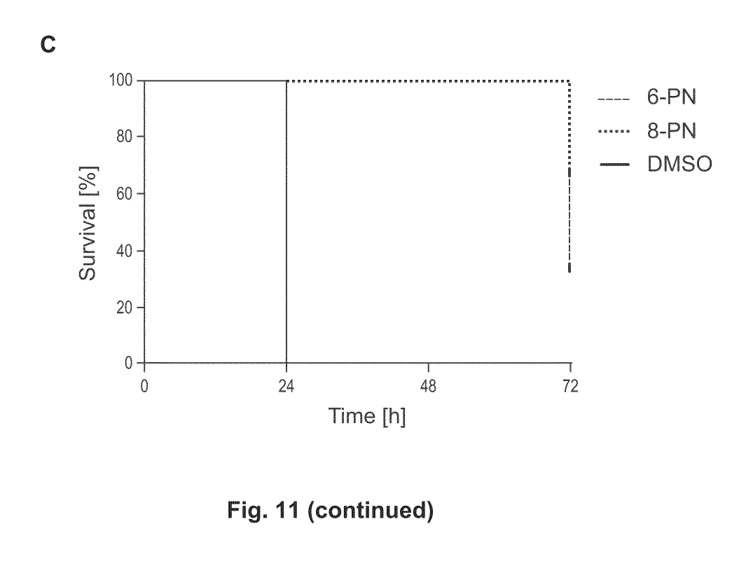

Since 6-PN and 8-PN had shown to exert antiproliferative effects on melanoma cells in vitro, it was asked whether such effects could be visualized in a more sophisticated human epidermal skin reconstruct. In this assay, keratinocytes and melanoma cells are seeded on top of a collagen matrix populated by fibroblasts. After 16 days a keratinizing stratified epithelium with a rudimentary corium is formed. Untreated control BLM cells (depicting 70-80% proliferation, determined by MIB1 immunhistochemistry) had formed a large tumor in the epidermal part of the reconstruct and had invaded the corium (FIG. 11A, upper panel). To determine the amount of proliferating cells, the number of MIB1 positive melanoma cells per power field in the epidermal part of the reconstruct at the epidermal-dermal border was counted (40× magnification, n=5 adjacent fields examined). Further, the number of melanoma cells invading the dermal part of the reconstruct was counted in the same high power fields. In reconstructs with untreated BLM cells, 103±15 MIB1 positive cells and 39±13 invasive cells were counted (FIG. 11A, upper panel). A treatment with 100 μM 6-PN significantly reduced the number of proliferating BLM cells and decreased the invasion: 27±6 MIB1 positive cells and 9±4 invasive cells (p<0.01, Student's t-test; FIG. 11A, middle panel). For 8-PN the effects were even more pronounced when compared to the untreated control cells: 16±4 MIB1 positive cells and 6±2 invasive cells (p<0.01, Student's t-test; FIG. 11A, lower panel). The epidermal skin reconstruct experiment was repeated with a second metastatic human melanoma cell line (LOXIMVI) to rule out cell line phenomenology in the particular skin reconstruct environment. In the untreated group, the LOXIMVI cells (aligned along the dermal-epidermal border and in prominent tumor cell nests in the dermal part of the aggregates) were 100% MIB1 positive (FIG. 11B). Both 6-PN and 8-PN treatments completely inhibited cell proliferation of LOXIMVI cells: only nests of dead LOXIMVI cells could be detected in the dermal part of the skin reconstructs (FIG. 11B).

Together, the antiproliferative effects observed in vitro could be reproduced and extended to an addition inhibition of BLM and LOXIMVI melanoma cell invasion in epidermal skin reconstructs.

2.7 In Vivo Toxicity Studies with 6-PN and 8-PN

Due to the detected cytotoxic effects on cancer cells and of melanocytes in vitro, the determination of in vivo embryotoxicity was of great interest. To this end, 2-day chicken embryos were treated for 24, 48 and 72 hours with 100 μM 6-PN or 100 μM 8-PN to monitor survival as readout for lethal embryotoxicity (FIG. 11C). Since the PN solvent used for the in vitro experiments (DMSO) negatively influenced the embryotoxicity assay and thus the determination of LD₅₀ values (7/7 embryos were dead 24 hours after the addition of the corresponding amount of DMSO; FIG. 11C), the PNs were now dissolved in a less toxic ethanol and PBS mixture instead of DMSO to minimize artificial side effects of the solvent for this in vivo experiment. Further, a pre-testing of the new solvent alone was performed. At a concentration of 500 μM of ethanol solvent, 6/7 embryos died within the first 24 hours and the remaining one after 48 hours, respectively. The 200 μM ethanol and PBS solvent mixture turned out to be the LD₅₀ concentration, while lower concentrations (100 μM and below) of the solvent were well tolerated. 6-PN and 8-PN were therefore tested at 100 μM, the concentration that had shown cytotoxic efficiency in the former assays. Little embryotoxic effects were observed 72 hours after treatment with PNs: 6/10 embryos survived after 72 hours after treatment with 6-PN, and 8/10 embryos after treatment with 8-PN (FIG. 11C). According to the very sensitive in vivo embryotoxicity assay concentrations of 100 μM 6-PN and 100 μM 8-PN were well tolerated.

### 3. Conclusion

The inventors were able to demonstrate on the basis of 19 different tumor cell lines in an impressive manner that a prenylflavonoid or prenylnaringenin (PN), respectively, in particular 6- and/or 8-prenylnaringenin (6-PN, 8-PN), is suited for the prophylaxis and/or treatment of a melanoma and/or a melanoma precursor and/or a skin and/or mucosa metastasis.

What is claimed is:

1. Pharmaceutical composition for the treatment or prophylaxis of a melanoma or a melanoma precursor or a skin or mucosa metastasis, comprising a prenylflavonoid as the active agent.

2. Pharmaceutical composition of claim 1, wherein the prenylflavonoid is a prenylnaringenin.

3. Pharmaceutical composition of claim 2, wherein the prenylnaringenin comprises 6- or 8-prenylnaringenin.

4. Pharmaceutical composition of claim 1, wherein the melanoma or the melanoma precursor are such of the skin.

5. Pharmaceutical composition of claim 4, wherein the melanoma is a melanoma skin or melanoma mucosa metastasis.

6. Pharmaceutical composition of claim 1 which is configured for a topical application.

7. Pharmaceutical composition of claim 6 which is configured for a topical application onto the skin.

8. Pharmaceutical composition of claim 7 comprising an application form which is selected from the group consisting of: ointment, cream, lotion, gel, paste, transdermal therapeutic system, foam, powder.

9. Pharmaceutical composition of claim 1, wherein it comprises an additional active agent which is effective against melanoma or melanoma precursors or skin or mucosa metastasis.

10. Pharmaceutical composition of claim 9, wherein the additional active agent is a necrosis inhibitor.

11. Pharmaceutical composition of claim 10, wherein the necrosis inhibitor is IM-54.

12. Method for the treatment or prophylaxis of a melanoma or a melanoma precursor or a skin or mucosa metastasis in a living being, comprising the following steps:
(1) Provision of the pharmaceutical composition of claim 1, and
(2) application of the pharmaceutical composition onto the skin, or the melanoma or the melanoma precursor or the skin metastasis or mucosa metastasis of the living being.
